(19) United States
(12) Patent Application Publication
(10) Pub. No.: US 2024/0366384 A1
(43) Pub. Date: Nov. 7, 2024

(54) AN IMPLANT HAVING AN IMPLANT SURFACE OF LAYERED COATINGS AND A METHOD THEREOF

(71) Applicant: WISSENKRAFT LABS PVT. LTD., Maharashtra (IN)

(72) Inventor: Chinmay Chandrashekhar Khare, Maharashtra (IN)

(21) Appl. No.: 18/683,187

(22) PCT Filed: Jul. 28, 2022
(86) PCT No.: PCT/IN2022/050682

§ 371 Date: Feb. 12, 2024

(30) Foreign Application Priority Data
Aug. 13, 2021 (IN) 202121036630

(51) Int. Cl. A61F 2/28; A61F 2/00

(52) U.S. Cl. CPC A61F 2/28; A61F 2/0077; A61F 2002/0081; A61F 2240/001; A61F 2310/00407; A61F 2310/00425

(57) ABSTRACT

An implant has an implant surface of layered coatings. A method of manufacturing is for an implant having an implant surface. The implant having an implant surface has a micro-pattern layer on the implant surface made by micro-machining, and a primary component layer deposited over the micro-pattern layer that promotes bio-integration of the implant. The micro-pattern layer has micro-trenches in predefined dimensions and arranged in a periodic array.

### CROSS-REFERENCE TO RELATED APPLICATIONS

This application is the U.S. National Stage of PCT/IN2022/050682 filed on Jul. 28, 2022, which claims priority to India Patent Application 202121036630 filed on Aug. 13, 2021, the entire content of both are incorporated herein by reference in their entirety.

### FIELD OF THE INVENTION

The invention relates broadly to the field of material sciences and coating technology. Particularly the invention relates to layered biocompatible, antimicrobial coatings and products made thereby. Still more particularly, the invention relates to a biocompatible & anti-microbial coating on implantable surfaces or any other medical products those possess bio-integrative surfaces and a process of coating thereof.

### BACKGROUND OF THE INVENTION

In numerous surgical interventions, an implant is fixed into a human body that is expected to perform a specific function. Typically, prosthetic implants serve as artificial joint elements that enable movements of replaced body parts. Likewise, various implants execute the desired function, which is incapacitated due to clinical reasons. It is imperative that an implant must be biocompatible, while the prosthetic-implants are expected to additionally promote osseointegration & demonstrate desired material strength.

The commonly used implant materials include Stainless Steel, Titanium alloys, Cobalt-Chromium alloys, Ceramics and Polymers. Some of these materials are susceptible to wear & corrosion, which may induce a risk of developing allergic reaction, inflammation etc. Furthermore, implant-associated & hospital-acquired infections remain a challenge with considerably high infection rates and in some cases even close to 30%. With conventional techniques like thermal spray methods (e.g., Plasma spray, flame spray, etc.) the prosthetic implant surfaces can be coated with biocompatible/bioactive coatings (e.g.: Hydroxyapatite, Titanium, or composite) which could enable efficient integration of the implant. However, such conventional thermal spray methods bring a drawback that both the substrate and coating are imperiled to oxidation, phase transformation and induce residual stresses, owing to the high temperature processes.

To address the challenge of infection, a recommended strategy involves integrating an antimicrobial (antibiotic or metal) agent in the coating that prevents bacterial growth and biofilm formation. One of the conventional methods to achieve antimicrobial surface of an implant comprises of a process involving anodizing the Ti-alloy (e.g.: Ti-6Al-4V) implant surface to develop cavities/pores. Subsequently, with electrochemical or dip-coating method the antimicrobial agent, such as Antibiotic or Silver, is loaded into the cavities/pores formed from anodizing treatment. One of the major constraining factors in this method is that the implant material should preferably be made of Ti or Ti-alloy. The other approach to achieve antimicrobial surface consists of coating the surface of an implant with osseo-conductive material loaded with Antibiotics (e.g.: HA loaded with Gentamicin). The shortfall of this method is associated with the risk of developing antimicrobial drug resistance for prolonged exposure. Therefore, typically the release of antibiotics is restricted maximum to <4-6 days. However, continuous activity inhibiting bacterial infection is of high clinical significance in the context of revision surgeries, immunocompromised patients & in tumor patients.

Simultaneously, it is an important challenge to restrict the antimicrobial activity in amount and in time in order to avert any cytotoxic effects & compromising the tissue compatibility.

Another problem that has been noticed in the case of joint replacement is aseptic loosening, that results in revision surgery. Here, efficient implant integration is a key challenge that needs to be addressed. Among the strategies developed, forming grooves or ridges on the implant surface is considered as an effective way that allows transfer of load/stresses on the trabecular axis of implant & thus prevents stress-shielding effect. However, in some of these cases, the grooves/ridges allow transfer of load on the implant, but lack in ideally promoting efficient cellular attachment & bone on-growth owing to the fact that the sizes of grooves or ridges are large in cell-size proportion. Correspondingly, the coating deposited on the implant surface does not exhibit surface features that could effectively promote bone on-growth.

Hence, there is a need for a layered coating of biocompatible, antimicrobial coating with a possibility to integrate specially designed micro-structured surface features that will enhance bio-integration for efficient implant integration & simultaneously display continuous antibacterial activity to inhibit bacterial growth & prevent biofilm formation.

The information disclosed in this background of the disclosure section is only for enhancement of understanding of the general background of the invention and should not be taken as an acknowledgement or any form of suggestion that this information forms the prior art already known to a person skilled in the art.

### SUMMARY OF THE INVENTION

In a non-limiting embodiment, an implant having an implant surface is disclosed. The implant surface comprises a micro-pattern layer and a primary component layer on the implant surface. The micro-pattern layer comprises micro-trenches made by micro-machining in predefined dimensions and arranged in a periodic array. Further, the primary component layer is deposited over the micro-pattern layer of micro-trenches. The implant surface promotes bio-integration of the implant.

In another embodiment, the micro-trenches made by micro-machining comprising the shape of one of hemi-circular, quasi-triangle, cross, isotoxal-star, oval, circular and square; in an arrangement comprising at least one of honeycomb-like or planar-hexagonal-closed-packed. In a non-limiting embodiment, the micro-trenches are of dimension of width in the range of 10 μm to 50 μm, depth of 50 μm to 500 μm and inter-pattern distance of 400 μm to 2000 μm.

In an embodiment, the primary component layer comprises at least one of titanium, titanium alloy, Titanium-Tantalum alloy, Magnesium alloy, Titanium-Zirconium alloy and/or combinations thereof as a primary component. The primary component layer is deposited by using a high-pressure cold-spray deposition technique. The high-pressure cold-spray deposition technique allows synthesizing of the primary component layer with porosity while retaining original phase of sprayed species of the primary component. In a non-limiting embodiment, the primary component, after being deposited, forms a thickness of layer from 70 μm to 800 μm.

In yet another embodiment, the implant having an implant surface further comprises an anti-microbial component layer deposited over the primary component layer using physical vapor deposition (PVD) technique. The anti-microbial component layer is configured for continuous release of anti-microbial component, from the anti-microbial component layer, to inhibit microbial growth and prevent colonization on the implant surface. In a non-limiting embodiment, the anti-microbial component comprises at least one of Silver (Ag), Gold (Au), Zinc (Zn), Platinum (Pt), Palladium (Pd), Iridium (Ir) and Copper (Cu), Nickel (Ni) or a combination thereof.

In another embodiment, the anti-microbial component layer is having a thickness of deposition in the range of 1 nm-500 nm. The thickness of deposition is regulated by tuning a duration of the deposition based on a rate of the deposition of the anti-microbial component on the implant surface. The thickness of deposition is determined based on the surface area of the implant in such a manner to prevent cytotoxicity caused due to the anti-microbial component.

In a non-limiting embodiment, a method of manufacturing an implant having an implant surface is disclosed. The method comprising the step of micro-machining to create a micropattern layer on the implant surface. The micro-pattern layer comprises of micro-trenches in predefined dimensions and arranged in a periodic array. The method further comprises the step of depositing a primary component layer over the micro-pattern layer. The implant surface promotes bio-integration of the implant.

In another embodiment, the method further comprises one or more intermittent steps of ultrasonic cleaning, surface cleaning and drying to remove organic/inorganic and other surface impurities, a further step of surface blasting with a blasting media to increase the surface roughness and a step of post-processing with nitrogen/compressed air blow, sterilization, and packaging/storage.

The foregoing summary is illustrative only and is not intended to be in any way limiting. In addition to the illustrative aspects, embodiments, and features described above, further aspects, embodiments, and features will become apparent by reference to the drawings and the following detailed description.

### Objects Of The Invention

One object of the invention is to provide an implant having an implant surface that promotes bio-integration of the implant upon implantation.

Another object of the invention is to prevent microbial growth on the implant surface and thereby promote better biocompatibility and bio-integration of the implant.

### BRIEF DESCRIPTION OF THE DRAWINGS

The accompanying drawings, which are incorporated in and constitute a part of this disclosure, illustrate exemplary embodiments and, together with the description, explain the disclosed embodiments. In the figures, the left-most digit(s) of a reference number identifies the figure in which the reference number first appears. The same numbers are used throughout the figures to reference like features and components. Some embodiments of implant and/or methods in accordance with embodiments of the present subject matter are now described, by way of example only, and with reference to the accompanying figures, in which:

FIG. 1 describes an exemplary environment 100 for an implant having an implant surface in accordance with an embodiment of the present invention;

FIG. 2(a) illustrates a cross-sectional view of the implant surface with micro-trenches in accordance with an embodiment of the present invention;

FIG. 2(b) illustrates a cross-sectional view of the implant surface with micro-trenches in accordance with an embodiment of the present invention;

FIG. 2(c) illustrates a cross-sectional view of the implant surface with primary component layer deposited over a micro-pattern layer in accordance with an embodiment of the present invention;

FIG. 2(d) demonstrates a top-view scanning electron microscopy (SEM) image of the implant surface upon the deposition of primary component layer over micro-pattern layer, in accordance with an embodiment of the present invention.

FIG. 2(e) illustrates a cross-sectional view of the implant surface upon deposition anti-microbial layer over the primary component layer and micro-pattern layer forming an anti-microbial micro-structured coating in accordance with an embodiment of the present invention;

FIG. 3 illustrates a flowchart describing a method of manufacturing an implant having an implant surface, in accordance with an embodiment of the present invention;

FIG. 4 shows a flowchart describing a method of manufacturing an implant having an implant surface with an anti-microbial component layer, in accordance with an embodiment of the present invention;

FIG. 5 depicts an embodiment of the method of manufacturing an implant having an implant surface along with intermediary steps used in sequential layer deposition in accordance with the disclosure of the present invention;

FIG. 6(a) depicts a graphical representation indicating significant increase in mineralization on an implant with aspects of anti-microbial micro-patterned layer in comparison with reference Titanium-VPS layer on the implant surface in accordance with an embodiment of the present invention;

FIG. 6(b) depicts a graphical representation indicating significant decrease in microbial activity on an implant with aspects of anti-microbial micro-patterned layer in comparison with reference Titanium-VPS layer on the implant surface in accordance with an embodiment of the present invention;

FIG. 7 depicts histopathological sections of bone samples indicating evidence of well-defined bone-formation at the bone-implant interface within the group that received implant with (a) antimicrobial micro-patterned layer and the group which received (b) reference Titanium vacuum plasma sprayed layer in accordance with an embodiment of the present invention;

FIG. 8 depicts micro-computed tomography of excised bone sample, wherein bony-growth can be noticed on the implant surface having antimicrobial micro-patterned layer indicating bio-integration in accordance with an embodiment of the present invention.

It should be appreciated by those skilled in the art that any block diagrams herein represent conceptual views of illustrative systems embodying the principles of the present subject matter. Similarly, it will be appreciated that any flow charts, flow diagrams, state transition diagrams, pseudo code, and the like represent various processes which may be substantially represented in computer readable medium and executed by a computer or processor, whether or not such computer or processor is explicitly shown.

### DETAILED DESCRIPTION OF THE INVENTION

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

In the following detailed description of the embodiments of the disclosure, reference is made to the accompanying drawings that form a part hereof, and in which are shown by way of illustration specific embodiments in which the disclosure may be practiced. These embodiments are described in sufficient detail to enable those skilled in the art to practice the disclosure, and it is to be understood that other embodiments may be utilized and that changes may be made without departing from the scope of the present disclosure. The following description is, therefore, not to be taken in a limiting sense.

Disclosed herein is an implant having an implant surface. The implant surface comprises a micro-pattern layer and a primary component layer on the implant surface. The micro-pattern layer comprises micro-trenches made by micro-machining in predefined dimensions and arranged in a periodic array. Further, the primary component layer is deposited over the micro-pattern layer of micro-trenches. The implant surface promotes bio-integration of the implant. The material for said implantable surfaces may selected from the group consisting of Stainless steel, Cobalt-chromium alloy, Cobalt-Chromium-Molybdenum alloy, Zirconium alloy, Ti-alloy, Ceramics, Polymers, and other materials which require a coating on the surface or any other combination thereof

In an aspect, implant surface comprises a micro-pattern layer of micro-trenches made by micro-machining comprising the shape of one of hemi-circular, quasi-triangle, cross, isotoxal-star, oval, circular and square; in an arrangement comprising at least one of honeycomb-like or planar-hexagonal closed-packed. In a non-limiting embodiment, the micro-trenches are of dimension of width in the range of 10 μm to 50 μm, depth of 50 μm to 500 μm and inter-pattern distance of 400 μm to 2000 μm.

In an aspect, the primary component layer comprises at least one of titanium and titanium alloy, Titanium-Tantalum alloy, Magnesium alloy, Titanium-Zirconium alloy and/or combinations thereof as a primary component. The primary component layer is deposited by using a high-pressure cold-spray deposition technique. The high-pressure cold-spray deposition technique allows synthesizing of the primary component layer with porosity while retaining original phase of sprayed species of the primary component. In a non-limiting embodiment, the primary component, after being deposited, forms a thickness of layer from 70 μm to 800 μm.

In an embodiment the said primary layer component layer is deposited using a high-pressure cold spray technique which allows synthesizing coatings with porosity, while retaining the original phase of the sprayed species. The coating is carried out with high pressure mode with pressure from 35 bar up to 50 bar, at a standoff distance of between 15 to 20 mm, with Argon, Nitrogen and/or Compressed Air as a carrier gas, preheated between 400° C. up to 800° C., and allowing adhesion of coating species by forming a condensed layer on the substrate surface.

In another aspect, the implant having an implant surface further comprises an anti-microbial component layer deposited over the primary component layer using physical vapor deposition (PVD) technique. The anti-microbial component layer is configured for continuous release of anti-microbial component, from the anti-microbial component layer, to inhibit microbial growth and prevent colonization on the implant surface. In a non-limiting embodiment, the anti-microbial component comprises at least one of Silver (Ag), Gold (Au), Zinc (Zn), Platinum (Pt), Palladium (Pd), Iridium (Ir) and Copper (Cu), Nickel (Ni) or a combination thereof.

In another aspect, the anti-microbial component layer is having a thickness of deposition in the range of 1 nm-500 nm. The thickness of deposition is regulated by tuning a duration of the deposition based on a rate of the deposition of the anti-microbial component on the implant surface. The thickness of deposition is determined based on the surface area of the implant in such a manner to prevent cytotoxicity caused due to the anti-microbial component.

In an aspect, said anti-microbial layer is synthesized by a process of physical vapour deposition with a partial pressure between 1 to 30 mbar in Argon atmosphere & sputtering with DC power/RF power between 10 W to 300 W under ambient temperature condition and may form thickness of layer from 1 to 500 nm.

In a non-limiting embodiment, a method of manufacturing an implant having an implant surface is disclosed. The method comprising the step of micro-machining to create a micropattern layer on the implant surface. The micro-pattern layer comprises of micro-trenches in predefined dimensions and arranged in a periodic array. The method further comprises the step of depositing a primary component layer over the micro-pattern layer. The implant surface promotes bio-integration of the implant.

In another embodiment, the method further comprises one or more intermittent steps of ultrasonic cleaning, surface cleaning and drying to remove organic/inorganic and other surface impurities, a further step of surface blasting with a blasting media to increase the surface roughness and a step of post-processing with nitrogen/compressed air blow, sterilization, and packaging/storage.

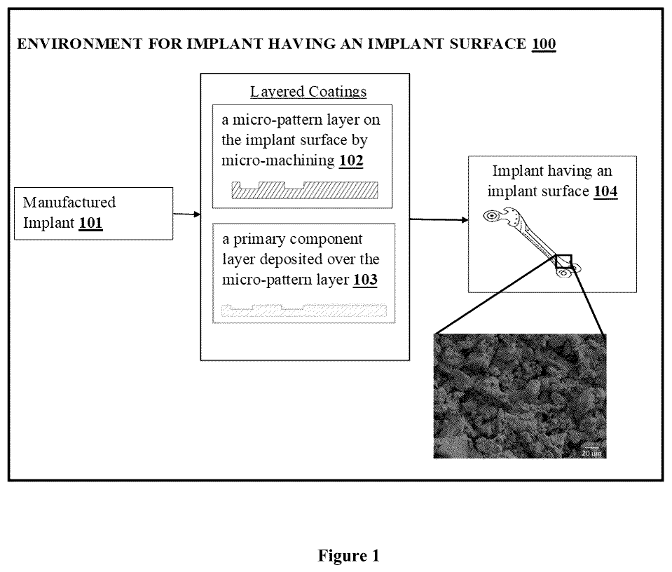

FIG. 1 describes an exemplary environment 100 for an implant having an implant surface in accordance with an embodiment of the present invention;

The present disclosure discloses an implant having an implant surface 104 of layered coatings that promotes bio-integration of the implant upon implantation. The environment 100 for an implant having an implant surface comprises a manufactured implant 101 with layered coatings to form the implant having implant surface 104. The layered coatings further comprise a micro-pattern layer 102 on the implant surface and a primary component layer 103 deposited over the micro-pattern layer. In an embodiment, the implant may be used in a body, for example human body to provide aid to or replace the structure or function of body, partially or completely. For example, the manufactured implant may be an orthopedic implant, dental implant, spinal implant, bionic etc . . . In an aspect of the invention, the manufactured implant 101 may be in a shape in accordance with the purpose/structure of the implant. For example, the manufactured implant 101 may be in the shape of a femur bone in case of an orthopedic implant.

In an embodiment an implant material 2 may be made of a biocompatible material, bioresorbable material or may selected from the group consisting of materials like Stainless steel, Cobalt-chromium alloy, Cobalt-Chromium-Molybdenum alloy, Zirconium alloy, Ti-alloy, Ceramics, Polymers, and other materials which require a coating on the surface or any other combination thereof, that further would comprise the implant surface 1. In another embodiment a partial implant where only a part or portion of the implant may be required to be implanted.

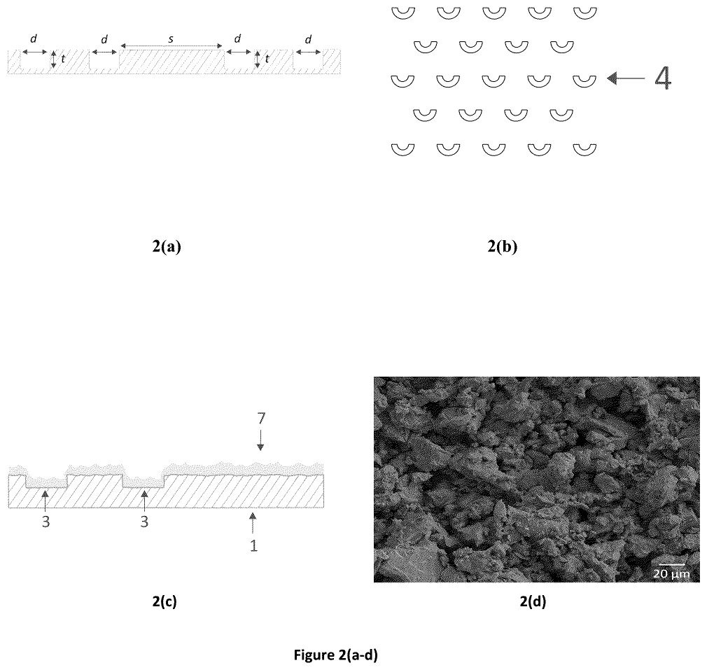

FIG. 2(a) illustrates a cross-sectional view of the implant surface with micro-trenches in accordance with an embodiment of the present invention.

In another embodiment of the present invention, a micro-pattern layer 102 is made on the implant surface by micro-machining tools and techniques. The micro-pattern layer 102 in accordance with the present application comprises micro-trenches 4 in predefined dimensions and arranged in a periodic array. Micro-patterning on the surface of the implant improves the surface morphology of the implant to enable better adhesion of subsequent layers and also acts as a scaffold for attachment and proliferation of cells for better bio-integration. However, the shape, dimensions like depth, width and inter-pattern distance of the micro-trenches 4 plays a significant role in enhancing these aspects. The geometric aspects such as width, depth and inter-pattern distance are denoted with ‘d’, ‘t’ and ‘s’ correspondingly (FIG. 2(a)). The micro-trenches 4 in accordance with the present disclosure are in a shape comprising of at least one of hemi-circular, quasi-triangle, cross, isotoxal-star, oval, circular and square in an arrangement comprising at least one of honeycomb-like or planar-hexagonal closed-packed arrangement. Further, the micro-trenches 4 are in a dimension range comprising of one or more of width 10 μm to 50 μm, depth 50 μm to 500 μm and inter-pattern distance 400 μm to 2000 μm. The shape and dimension of the micro-trenches 4 in an embodiment may be predefined in an manner based on the type of the implant, the surface area on the implant that requires bio-integration, the region of the implant on the body and the cell-size proportion etc. For example, a hemi-circular shaped micro-pattern in a planar hexagonal closed-packed arrangement may used on an orthopedic implant to promote osseo-integration (refer FIG. 2(b)).

FIG. 2(c) illustrates a cross-sectional view of the implant surface with primary component layer deposited over a micro-pattern layer in accordance with an embodiment of the present invention;

In an embodiment of the present invention, the implant having an implant surface 1 has a primary component layer 103 over the micro-pattern layer 102. The commonly used implant materials include Stainless Steel, Titanium alloys, Cobalt-Chromium alloys, Ceramics and Polymers. Some of these materials are susceptible to wear & corrosion, which may induce a risk of developing allergic reaction, inflammation etc. Furthermore, implant-associated & hospital-acquired infections remain a challenge with considerably high infection rates and in some cases even close to 30%. With conventional techniques like thermal spray methods (e.g. Plasma spray, flame spray, etc) for deposition of primary component layer 103 the prosthetic implant surfaces 1 can be coated with biocompatible/bioactive coatings (e.g.: Hydroxyapatite, Titanium or composite) which could enable efficient integration of the implant. However, such conventional thermal spray methods bring a drawback that both the implant surface 1 and coating layer are imperiled to oxidation, phase transformation and induce residual stresses, owing to the high temperature processes.

Therefore, in accordance with an embodiment of the present disclosure discloses the primary component layer 103 on implant surface 1 is deposited by cold spray method which allows synthesis of coatings with porosity, while retaining the original phase of the sprayed species. The porosity of the deposited primary component layer 103 further enhances the attachment and proliferation of the cell over the implant surface 1 for better bio-integration. FIG. 2(d) demonstrates a top-view scanning electron microscopy (SEM) image of the implant surface 1 upon the deposition of primary component layer 103 over micro-pattern layer 102, in accordance with an embodiment of the present invention.

In this case, the coated layered implant surface 1 essentially performs two functions:
releases silver ions, in effective amount, which inhibits the bacterial/microbial growth and prevents colonization. As the time progresses the silver content within the film may exhaust continuously, thereby achieving continuous antimicrobial effect.
Simultaneously, the micro-patterned layer in addition to the rough morphology of the primary component layer 103 allows cell attachment & rapid cell growth. This significantly improves the quality of bio-integration while simultaneously reducing the risk of infection & microbial colonization. Moreover, the Ti-coating will be biocompatible and does not corrode in body fluids like for example synovial fluid in case of knee implant.

FIG. 2(e) illustrates a cross-sectional view of the implant surface upon deposition anti-microbial layer over the primary component layer and micro-pattern layer forming an anti-microbial micro-structured coating in accordance with an embodiment of the present invention;

In addition to the layers of micro-pattern layer 102 and primary component layer 103, the bio-integration of the implant having implant surface 1 may be enhanced by an anti-microbial layer in accordance to an embodiment. The deposition of the anti-microbial layer prevents the formation of microbial film which may leading to infection at the interface of implant site in the body and the bodily fluids. FIG. 2(e) illustrates an embodiment with an anti-microbial component for example like silver deposited over the primary component layer 103. This layer upon implantation of the implant at the implant site in the body gradually release ions preventing or reducing significantly any microbial activity at the implant site. Therefore, the anti-microbial component layer is configured for continuous release of anti-microbial component, from the anti-microbial component layer, to inhibit microbial growth and prevent colonization on the implant surface 1. The anti-microbial component comprises at least one of Silver (Ag), Gold (Au), Zinc (Zn), Platinum (Pt), Palladium (Pd), Iridium (Ir) and Copper (Cu), Nickel (Ni), or a combination thereof having a thickness of deposition in the range of 1 nm-500 nm. It is observed that the anti-microbial components may cause cyto-toxicity over corresponding threshold values for the same. To avoid such scenarios the thickness of deposition may be regulated by tuning a duration of the deposition based on a rate of the deposition of the anti-microbial component on the implant surface 1, and the thickness of deposition is determined based on the surface area of the implant in such a manner to prevent cytotoxicity caused due to the anti-microbial component. For example the thickness of the anti-microbial component layer over an implant with large surface area may be less in comparison to the thickness of the anti-microbial component layer over an implant with small surface area. Physical vapour deposition (PVD) is a process used to produce a metal vapour that can be deposited on electrically conductive materials as a thin, highly adhered pure metal or alloy coating. Using of PVD technique for deposition of anti-microbial layer provides unique control over the thickness of the layer deposited in nanoscale calculated based on the rate of deposition.

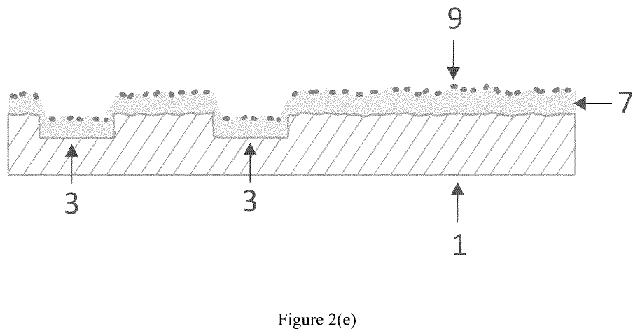

FIG. 3 illustrates a flowchart describing a method of manufacturing an implant having an implant surface in accordance with an embodiment of the present invention.

As illustrated in FIG. 3, the method of manufacturing an implant having an implant surface 1 is described.

The order in which the method is described is not intended to be construed as a limitation, and any number of the described method blocks can be combined in any order to implement the method.

At block 301, the method 300 may include micro-machining to create a micropattern layer on the implant surface 1. The micro-pattern layer 102 comprises of micro-trenches 4 in predefined dimensions and arranged in a periodic array. The micro-trenches 4 are in a shape comprising of at least one of hemi-circular, quasi-triangle, cross, isotoxal-star, oval, circular and square in a honeycomb-like or planar-hexagonal closed-packed-like arrangement. In an embodiment the material of the said implant surface Is may be selected from the group consisting of Stainless steel, Cobalt-chromium alloy, Cobalt-Chromium-Molybdenum alloy, Zirconium alloy, Ti-alloy, Ceramics, Polymers and other materials which require a coating on the surface for enhancing biocompatibility. The micro-machining comprises creating micro-trenches 4 in a dimension ranges comprising of width 10 μm to 50 μm, depth 50 μm to 500 μm and inter-pattern distance 400 μm to 2000 μm.

At block 302, the method 300 may include depositing a primary component layer 103 over the micro-pattern layer 102. The primary component layer 103 comprises at least one of titanium and titanium alloy. Depositing the primary component layer 103 is performed by using a high-pressure cold-spray deposition technique. The cold spray deposition technique allows synthesizing of the primary component layer 103 with porosity, while retaining original phase of sprayed species of the primary component. Depositing the primary component forms a thickness of layer from 70 μm to 800 μm. The implant surface 1 promotes bio-integration of the implant. In an embodiment, the primary component of the coating composition is a metallic agent comprising either Titanium (Ti) or Titanium-alloy. In one embodiment, primary component of the coating composition is in the form of powder (particle size<65-90 μm) deposited by spray deposition and may form a thickness of layer ranging from 70 μm up to 500 μm.

The method 300 may further include depositing an anti-microbial component layer over the primary component layer 103 using physical vapor deposition (PVD) technique, The anti-microbial component layer is configured for continuous release of the anti-microbial component from the anti-microbial component layer, to inhibit microbial growth and prevent colonization on the implant surface 1. The anti-microbial component comprises at least one of Silver (Ag), Gold (Au), Zinc (Zn), Platinum (Pt), Palladium (Pd), Iridium (Ir), Copper (Cu) and Nickel (Ni) or a combination thereof.

The anti-microbial component layer has a thickness of deposition in the range of 1 nm-500 nm. The thickness of deposition is regulated by tuning duration of deposition based on a rate of deposition of the anti-microbial component on the implant surface 1. The thickness of deposition is determined based on the surface area of the implant in such a manner to prevent cytotoxicity caused due to anti-microbial component

The method 300 may further include one or more intermittent steps of ultrasonic cleaning, surface cleaning and drying to remove organic/inorganic and other surface impurities, a further step of surface blasting with a blasting media to increase the surface roughness and a step of post-processing with nitrogen/compressed air blow, sterilization and packaging/storage.

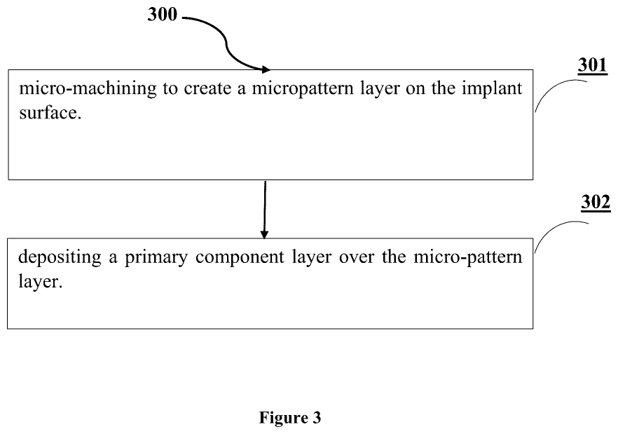

FIG. 4 illustrates a flowchart describing a method of manufacturing an implant having an implant surface in accordance with another embodiment of the present invention.

As illustrated in FIG. 4, the method of manufacturing an implant having an implant surface 1 is described.

At block 401, the method 400 may include micro-machining to create a micropattern layer on the implant surface 1. The micro-pattern layer 102 comprises of micro-trenches 4 in predefined dimensions and arranged in a periodic array.

At block 402, the method 400 may include depositing a primary component layer 103 over the micro-pattern layer 102, the implant surface 1 promotes bio-integration of the implant.

At block 403, the method 400 may include depositing an anti-microbial component layer over the primary component layer 103 using physical vapor deposition (PVD) technique, wherein the anti-microbial component layer is configured for continuous release of the anti-microbial component from the anti-microbial component layer, to inhibit microbial growth and prevent colonization on the implant surface 1.

The method 400 may further include depositing an anti-microbial component layer over the primary component layer 103 using physical vapor deposition (PVD) technique, The anti-microbial component layer is configured for continuous release of the anti-microbial component from the anti-microbial component layer, to inhibit microbial growth and prevent colonization on the implant surface 1. The anti-microbial component comprises at least one of Silver (Ag), Gold (Au), Zinc (Zn), Platinum (Pt), Palladium (Pd), Iridium (Ir), Copper (Cu) and Nickel (Ni) or a combination thereof

The anti-microbial component layer has a thickness of deposition in the range of 1 nm-500 nm. The thickness of deposition is regulated by tuning a duration of deposition based on a rate of deposition of the anti-microbial component on the implant surface 1. The thickness of deposition is determined based on the surface area of the implant in such a manner to prevent cytotoxicity caused due to anti-microbial component

The method 400 may further include one or more intermittent steps of ultrasonic cleaning, surface cleaning and drying to remove organic/inorganic and other surface impurities, a further step of surface blasting with a blasting media to increase the surface roughness and a step of post-processing with nitrogen/compressed air blow, sterilization and packaging/storage.

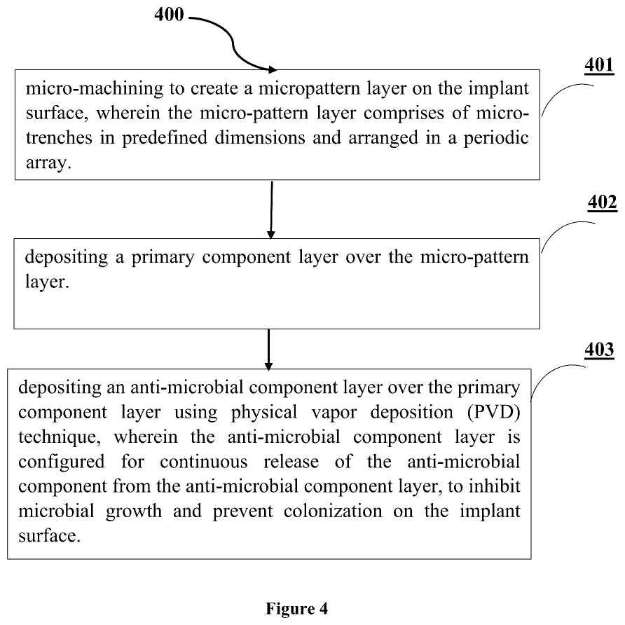

FIG. 5 depicts an embodiment of the method of manufacturing an implant having an implant surface 1 along with intermediary steps used in sequential layer deposition in accordance with the disclosure of the present invention.

The method of manufacturing an implant having an implant surface 1 along with intermediary steps used in sequential layer deposition includes one or more intermittent steps of ultrasonic cleaning, surface cleaning and drying to remove organic/inorganic and other surface impurities, a further step of surface blasting with a blasting media to increase the surface roughness and a step of post-processing with nitrogen/compressed air blow, sterilization and packaging/storage. The order in which the method is described is not intended to be construed as a limitation, and any number of the described method steps can be combined in any order to implement the method.

### Examples

### Example 1: Preparation of Micro-trenches on the Implant Surface 1

Ultrasonic cleaning & drying: Firstly, the implant material is subjected to ultrasonic cleaning & drying of implant surface 1 to remove organic/inorganic & other surface impurities. The material for said implantable surfaces may selected from the group consisting of Stainless steel, Cobalt-chromium alloy, Cobalt-Chromium-Molybdenum alloy, Zirconium alloy, Ti-alloy, Ceramics, Polymers and other materials which require a coating on the surface or any other combination thereof

Micro-machining: The cleaned surface is then subjected to micro-machining on the surface to create periodic micro-trenches 4. The micro-pattern layer 102 on the implant surface 1 is created using micro-machining tool 2 to form micro-trenches 4. The coating process includes pre-patterning of substrate surface to obtain identical periodic micro-trenches 4 and involve the processing technique of micromachining may include but not limited to laser patterning, photolithography, micro-imprinting, selective laser melting/additive manufacturing, microstamping, etching or any other and combinations thereof. The micro-trenches 4 are made in such a manner to have a defined shape & dimension. The geometric aspects such as width, depth and inter-pattern distance are denoted with ‘d’, ‘t’ and ‘s’ (FIG. 2(a)). The micro-trenches 4 are arranged in a periodic configuration such as planar-hexagonal-closed-packed arrangement 4 (FIG. 2b) or honeycomb like arrangement. The shape of the micro-trenches 4 comprises of at least one of hemi-circular, quasi-triangle, cross, isotoxal-star, oval, circular and square. The micro-trenches 4 made by micro-machining has a dimension of width ranging from 10 μm to 50 μm, depth ranging from 50 μm to 500 μm and inter-pattern distance ranging from 400 μm to 2000 μm. The step further includes an ultrasonic surface cleaning, rinsing & drying of implantable surface upon micro-machining to eliminate surface impurities.

Surface blasting: Additionally, a surface blasting with blasting-media onto the implant surface 1 using a blasting apparatus 5 is carried out to increase the surface roughness and is done by blasting the surface with Silicon Carbide (SiC), Alumina (Al2O3), Silicon dioxide (SiO2) powder or any other combinations thereof. Blasting is followed by the step of ultrasonic cleaning & drying of implant surface 1 upon surface blasting to remove organic/inorganic & other surface impurities.

### Example 2: Deposition of Primary Component Layer on the Implant Surface 1 With Micro-Trenches 4

The method may include the step of deposition of primary component on the implant surface 1. The said deposition of primary coating on substrate surface is carried out by the coating techniques of high-pressure cold spray 6. The coating is carried out with high pressure mode with pressure from 35 bar up to 50 bar, at a standoff distance of between 15 to 20 mm, with Argon, Nitrogen and/or Compressed Air as a carrier gas, preheated between 400° C. up to 800° C., and allowing adhesion of coating species by forming a condensed layer on the substrate surface. This high-pressure cold spray 6 deposition allows synthesizing coatings with porosity, while retaining the original phase of the sprayed species. The implant with the surface coated with the primary component layer 7, 103 is further subjected to surface cleaning & drying of implant surface 1 to eliminate surface impurities.

Further the deposition of primary component layer 7, 103 on micro-patterned surface 3 may also be carried out by the coating techniques selected from the group consisting of thermal spray, vacuum plasma spray, atmospheric plasma spray, detonation spray, or any other combination thereof. Using techniques like high-pressure cold spray deposition for deposition of primary component layer overcomes the need for a bonding material between the implant surface and the primary component, thereby reducing the comparative cost of production significantly.

### Example 3: Depositing of Anti-Microbial Component Layer

The method of depositing anti-microbial component layer 9 on implantable surface includes depositing said antimicrobial Ag layer. The antimicrobial agent to be coated on the implantable surfaces have Ag concentration ranging from 0.1 at. % up to 45 at. %. As a noble metal Silver (Ag) (E0=+.80 V) does not corrode in water, it gradually releases silver ions (Ag+) either in direct contact or by release of free ions Ag metal is known to exhibit antimicrobial function. These metal ions can react with a variety of microbial structures leading to damage to the cell wall, membranes and intracellular metabolic activities. This circumvents or inhibits microbial growth and prevents colonization.

It is observed that the anti-microbial components may cause cyto-toxicity over corresponding threshold values for the same. To avoid such scenarios the thickness of deposition may be regulated by tuning a duration of the deposition based on a rate of the deposition of the anti-microbial component on the implant surface 1, and the thickness of deposition is determined based on the surface area of the implant in such a manner to prevent cytotoxicity caused due to the anti-microbial component. For example, the thickness of the anti-microbial component layer over an implant with large surface area may be less in comparison to the thickness of the anti-microbial component layer over an implant with small surface area. Physical vapour deposition (PVD) is a process used to produce a metal vapour that can be deposited on electrically conductive materials as a thin, highly adhered pure metal or alloy coating thereby overcoming the need for a bonding material between the primary component layer 103 and the anti-microbial component layer 9, thereby reducing the comparative cost of production significantly. Using of PVD technique for deposition of anti-microbial layer provides unique control over the thickness of the layer deposited in nanoscale calculated based on the rate of deposition. The antimicrobial Ag layer is synthesized by a process of physical vapour deposition with a partial pressure between 1 to 30 mbar in Argon atmosphere & sputtering with DC power/RF power between 10 W to 300 W under an ambient temperature condition to obtain a thickness of layer from 1 to 500 nm.

Deposition of anti-microbial component layer 9 on implantable surface is further be followed by steps of cleaning and sterilization of coated implant surface 1.

### Example 4: Post-Processing

The post-processing step upon the deposition of various layers include one or more of post-processing steps like for example blowing nitrogen/compressed air to clean the implant surface 1, sterilization & packaging/storage of the implant with the implant surface 1.

### Example 5

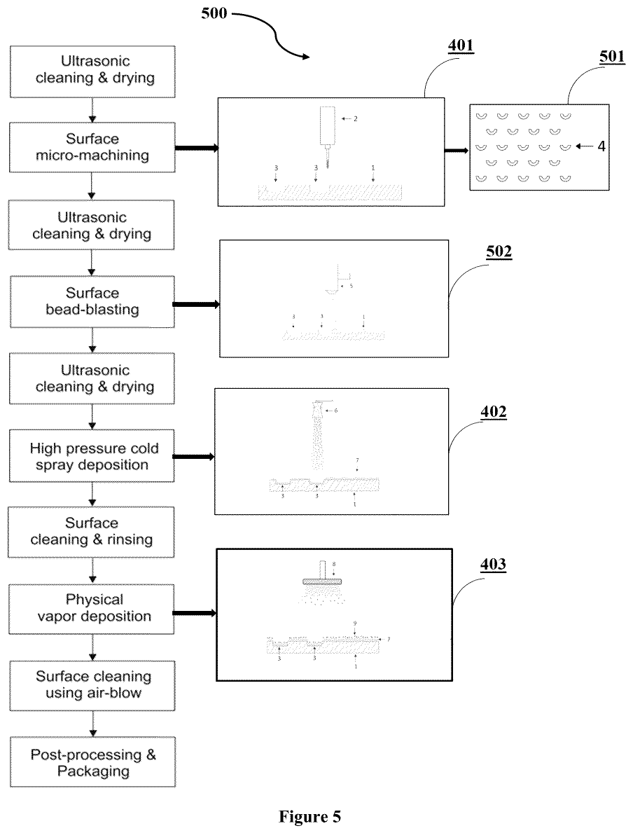

The ability of the implant surface 1 to inhibit microbial growth was assessed by setting up antibacterial activity assay by incubating bacterial inoculums on the surface of the coupon discs. After 24 hours of incubation the bacterial inoculums were recovered & plated in petri dishes for colony counting purpose. The implant surface 1 comprising of a micro-pattern layer 102 and a primary component layer 7,103 likely comprising of Titanium (FIG. 2(c)) as well as antimicrobial component likely comprising of Ag 9 yielding antimicrobial micro-structured layer was synthesized on Ti₆Al₄V coupon discs of diameter 10 mm & thickness 3 mm for performing biochemical in-vitro assays and as a reference, Ti₆Al₄V coupon discs of similar shape & dimension with Titanium layer deposited with vacuum plasma spray method were used. FIG. 6(a) depicts a graphical representation indicating significant increase in mineralization on an implant with aspects of anti-microbial component layer 9 and a micro pattern layer on the implant surface 1 in accordance with an embodiment of the present invention. It depicts that mineralization activity at two different timepoints on antimicrobial micro-structured coating & reference Titanium-VPS coating (* indicates significant difference with p<0.5).

The antimicrobial activity was examined against gram-negative Escherichia coli bacteria and gram-positive Staphylococcus aureus. The antimicrobial micro-structured layer demonstrated four log reduction & two log reduction values in comparison to reference Titanium vacuum plasma spray layer (VPS), for Escherichia coli and Staphylococcus aureus bacteria, respectively (FIG. 6(b) depicts a graphical representation indicating significant decrease in microbial activity on an implant with aspects of anti-microbial component layer 9 and a micro pattern layer on the implant surface 1 in accordance with an embodiment of the present invention.

### Example 6

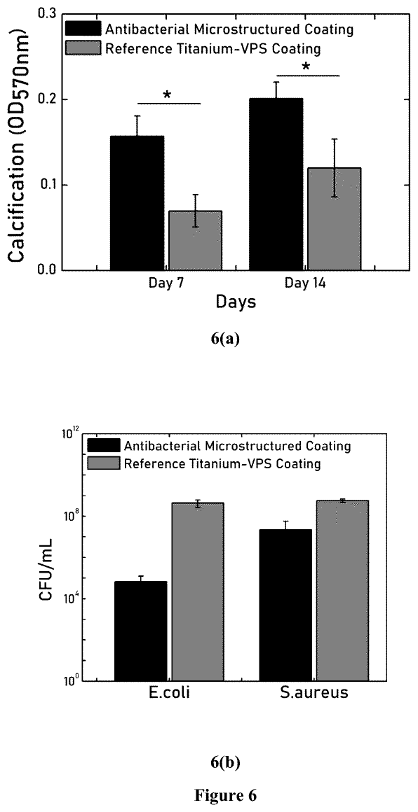

To validate safety & assess biological response of the implant having implant surface 1 with antimicrobial micro-structured layer an in-vivo implantation study was performed. As a test system New Zealand White Rabbits were selected as an appropriate species for a 13 weeks implantation study. The study design comprised of two groups which received cylindrical implants with antimicrobial micro-structured layer and reference Titanium vacuum plasma sprayed layer. Under sterile conditions, after making incision the implants were inserted in the tibia bones of New Zealand White Rabbits and wounds were sutured. After 13 weeks of implantation, the rabbits were humanely sacrificed and bone specimen containing implants were excised. Following decalcification treatment, histopathological sections of the bone samples were prepared. Implantation did not cause any adverse effects including irritation or inflammatory reaction. Gross histopathological observations of bone samples revealed comparable microanatomical tissue organization within both groups. Histopathology sections demonstrated evidence of well-defined bone-formation at the bone-implant interface within the group that received implant with antimicrobial micro-structured layer and the group which received reference Titanium vacuum plasma sprayed layer (Refer FIG. 7 depicting the histopathological sections of bone samples indicating evidence of well-defined bone-formation at the bone-implant interface within the group that received implant with (a) antimicrobial micro-patterned layer and the group which received (b) reference Titanium vacuum plasma sprayed layer in accordance with an embodiment of the present invention).

### Example 7

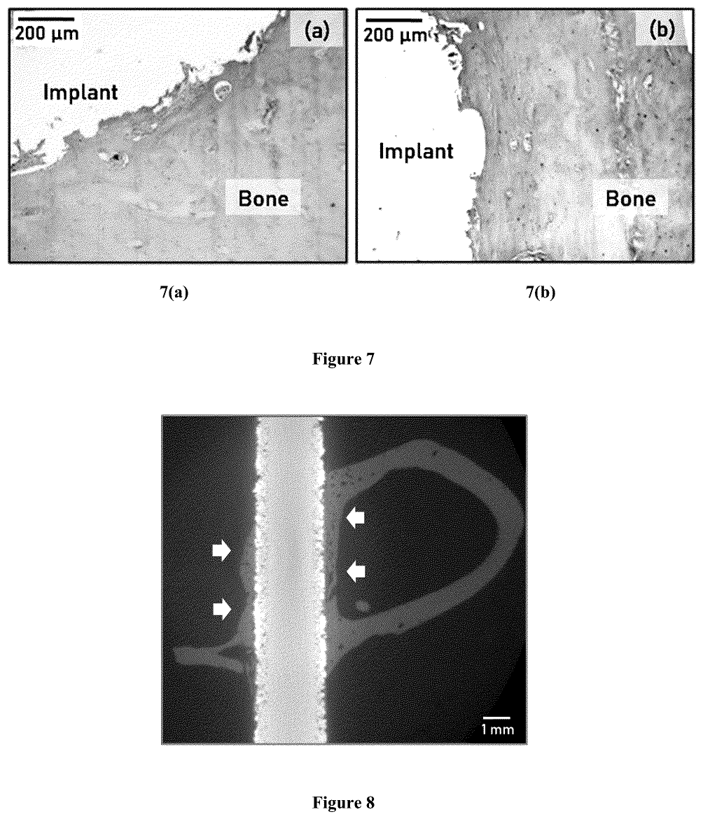

Bio-integration of implant was assessed by performing micro-computed tomography of excised bone sample, which received implant with antimicrobial micro-structured layer, from the in-vivo study as described above. The implant surface 1 with antimicrobial micro-structured layer allowed bony-growth (as marked white arrows) and thereby achieving osseous integration of the implant (Refer FIG. 8 depicting micro-computed tomography of excised bone sample, wherein bony-growth can be noticed on the implant surface 1 having antimicrobial micro-patterned layer indicating bio-integration in accordance with an embodiment of the present invention). A description of an embodiment with several components in communication with each other does not imply that all such components are required. On the contrary, a variety of optional components are described to illustrate the wide variety of possible embodiments of the invention.

When a single device or article is described herein, it will be clear that more than one device/article (whether they cooperate) may be used in place of a single device/article. Similarly, where more than one device or article is described herein (whether they cooperate), it will be clear that a single device/article may be used in place of the more than one device or article, or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself. Finally, the language used in the specification has been principally selected for readability and instructional purposes, and it may not have been selected to delineate or circumscribe the inventive subject matter. It is therefore intended that the scope of the invention be limited not by this detailed description, but by any claims that issue on an application based here on. Accordingly, the embodiments of the present invention are intended to be illustrative, but not limiting, of the scope of the invention, which is set forth in the following claims.

While various aspects and embodiments have been disclosed herein, other aspects and embodiments will be apparent to those skilled in the art. The various aspects and embodiments disclosed herein in the examples are for purposes of illustration and are not intended to be limiting, with the true scope and spirit being indicated in the claims.

1. An implant having an implant surface comprising:
a micro-pattern layer on the implant surface by micro-machining, wherein the micro-pattern layer comprises micro-trenches in predefined dimensions and arranged in a periodic array; and
a primary component layer deposited over the micro-pattern layer; wherein the implant surface promotes bio-integration of the implant.

2. The implant as claimed in claim 1, wherein the primary component layer comprises at least one of titanium and titanium alloy, Titanium-Tantalum alloy, Magnesium alloy, Titanium-Zirconium alloy and/or combinations thereof as a primary component, and wherein the primary component layer is deposited by using a high-pressure cold-spray deposition technique, wherein the cold spray deposition technique allows synthesizing of the primary component layer with porosity while retaining original phase of sprayed species of the primary component, wherein the primary component, after being deposited, forms a thickness of layer from 70 μm to 800 μm.

3. The implant as claimed in claim 1, wherein the micro-trenches are in a shape comprising of at least one of hemi-circular, quasi-triangle, cross, isotoxal-star, oval, circular and square in an arrangement comprising at least one of honeycomb-like or planar-hexagonal closed-packed arrangement, wherein the micro-trenches are in a dimension range comprising of one or more of width 10 μm to 50 μm, depth 50 μm to 500 μm and inter-pattern distance 400 μm to 2000 μm.

4. The implant as claimed in claim 1, further comprising an anti-microbial component layer deposited over the primary component layer using physical vapor deposition technique, wherein the anti-microbial component layer is configured for continuous release of anti-microbial component, from the anti-microbial component layer, to inhibit microbial growth and prevent colonization on the implant surface;
wherein the anti-microbial component comprises at least one of Silver, Gold, Zinc, Platinum, Palladium, Iridium and Copper, Nickel or a combinations thereof.

5. The implant as claimed in claim 4, wherein the anti-microbial component layer is having a thickness of deposition in the range of 1 nm-500 nm, wherein the thickness of deposition is regulated by tuning a duration of the deposition based on a rate of the deposition of the anti-microbial component on the implant surface, and wherein the thickness of deposition is determined based on the surface area of the implant in such a manner to prevent cytotoxicity caused due to the anti-microbial component.

6. A method of manufacturing an implant having an implant surface, the method comprising the steps of:
micro-machining to create a micro-pattern layer on the implant surface, wherein the micro-pattern layer comprises of micro-trenches in predefined dimensions and arranged in a periodic array: and
depositing a primary component layer over the micro-pattern layer;
wherein the implant surface promotes bio-integration of the implant.

7. The method as claimed in claim 6, wherein the method further comprises one or more intermittent steps of ultrasonic cleaning, surface cleaning and drying to remove organic/inorganic and other surface impurities, a further step of surface blasting with a blasting media to increase the surface roughness and a step of post-processing with nitrogen/compressed air blow, sterilization and packaging/storage.

8. The method as claimed in claim 6, wherein the primary component layer comprises at least one of titanium and titanium alloy, Titanium-Tantalum alloy, Magnesium alloy, Titanium-Zirconium alloy and/or combinations thereof as a primary component, and wherein depositing the primary component layer is performed by using a high-pressure cold-spray deposition technique, wherein the cold spray deposition technique allows synthesizing of the primary component layer with porosity, while retaining original phase of sprayed species of the primary component,
wherein depositing the primary component forms a thickness of layer from 70 μm to 800 μm.

9. The method as claimed in claim 6, wherein the micro-trenches are in a shape comprising of at least one of hemi-circular, quasi-triangle, cross, isotoxal-star, oval, circular and square in a honeycomb-like or planar-hexagonal closed-packed-like arrangement,
wherein the micro-machining comprises creating micro-trenches in a dimension ranges comprising one or more of width 10 μm to 50 μm, depth 50 μm to 500 μm and inter-pattern distance 400 μm to 2000 μm.

10. The method as claimed in claim 6, further comprising depositing an anti-microbial component layer over the primary component layer using physical vapor deposition technique, wherein the anti-microbial component layer is configured for continuous release of the anti-microbial component from the anti-microbial component layer, to inhibit microbial growth and prevent colonization on the implant surface,
wherein the anti-microbial component comprises at least one of Silver, Gold, Zinc, Platinum, Palladium, Iridium, Copper and Nickel or a combination thereof.

11. The method as claimed in claim 10, wherein the anti-microbial component layer is having a thickness of deposition in the range of 1 nm-500 nm, wherein the thickness of deposition is regulated by tuning a duration of deposition based on a rate of deposition of the anti-microbial component on the implant surface, wherein the thickness of deposition is determined based on the surface area of the implant in such a manner to prevent cytotoxicity caused due to anti-microbial component.
